(19) United States
(12) Patent Application Publication
(10) Pub. No.: US 2020/0030154 A1
(43) Pub. Date: Jan. 30, 2020

(54) SYSTEM AND METHOD FOR ANTIMICROBIAL NASAL CAVITY BANDAGE DRESSING

(71) Applicant: Jeremy CHRISTIAN RAMIREZ, San Jose, CA (US)

(72) Inventor: Jeremy CHRISTIAN RAMIREZ, San Jose, CA (US)

(21) Appl. No.: 16/491,302

(22) PCT Filed: Jan. 29, 2018
(86) PCT No.: PCT/US18/15718

§ 371 Date: Sep. 5, 2019

Related U.S. Application Data
(60) Provisional application No. 62/472,384, filed on Mar. 16, 2017.

(51) Int. Cl. A61F 13/12; A61F 13/00

(52) U.S. Cl. CPC A61F 13/126; A61F 13/00063; A61F 2013/00714; A61F 2013/00476; A61F 2013/00846

(57) ABSTRACT

A nasal cavity bandage dressing and method of use thereof, including a bandage made from a waterproof, antimicrobial, and adhesive material; and a multi-layered, absorbent sterile dressing affixed to the bandage. The bandage affixed with the non-adherent multi-layered absorbent sterile dressing is configured to fit within a nasal cavity.

### CROSS REFERENCE TO RELATED DOCUMENTS

The present invention claims priority to U.S. Provisional Patent Application Ser. No. 62/472,384 of Jeremy Christian RAMIREZ, entitled “SYSTEM AND METHOD FOR ANTIMICROBIAL NASAL CAVITY BANDAGE DRESSING,” filed on 16 Mar. 2017, the entire disclosure of which is hereby incorporated by reference herein.

### BACKGROUND OF THE INVENTION

### Field of the Invention

The present invention generally relates to systems and methods for protective bandages, and more particularly to a method and system for a bandage dressing that can be uniquely applied to a mucous membrane, and the like, within a nasal cavity, and the like.

### Discussion of the Background

In recent years, excessive nasal discharge, and the like, has become a common health problem, wherein when nasal discharge occurs, it can oftentimes become a frustrating experience. However, existing systems and methods are not robust with respect to providing for a protective product that address these and other problems with existing systems and methods for protective bandages.

### SUMMARY OF THE INVENTION

Therefore, there is a need for a method and system that addresses the above and other problems. The above and other problems are addressed by the illustrative embodiments of the present invention, which provide a nasal cavity bandage, for example, made of clear or translucent, waterproof, antimicrobial adhesive material, and the like, that is safe for direct application to the nasal mucous membrane, and the like. Attached to the bandage is a non-adherent multi-layered absorbent sterile dressing. The dressing is non-adherent to the mucous membrane and coated with a porous polymer, for example, such as aloe vera, and the like, to allow for soothing comfort and gentle removal. The nasal cavity bandage dressing is tough, but malleable, and thus, capable of lining the inner contours of the nose with ease of placement where it can block excessive and continuous nasal discharge caused by various conditions, for example, including a cold or flu virus, seasonal allergies, dry climate, spicy foods, and the like. Accordingly, the nasal cavity bandage dressing, can include multi-layers of non-adherent dressing. Advantageously, the nasal cavity bandage dressing is of a compact and clear or translucent design that does not obstruct breathing, is not easily visible, can easily be applied with skin safe adhesive material to a nasal mucous membrane, and generally overcomes many of the numerous problems noted with respect to excessive nasal discharge.

Accordingly, in illustrative aspects of the present invention there is provided a nasal cavity bandage dressing and method of use thereof, including a bandage made from a waterproof, antimicrobial, and adhesive material; and a multi-layered, absorbent sterile dressing affixed to the bandage. The bandage affixed with the non-adherent multi-layered absorbent sterile dressing is configured to fit within a nasal cavity.

With the nasal cavity bandage dressing and use thereof, an outer surface of the bandage is coated with an antibacterial agent, including Benzalkonium Chloride 0.1% to protect against risk of infection associated with placement within the nasal cavity.

With the nasal cavity bandage dressing and use thereof, the bandage is waterproof, allowing for sustained placement within a moist environment of the nasal cavity.

With the nasal cavity bandage dressing and use thereof, the bandage is made from a tough, but malleable material that allows for elasticity without being flimsy so as not to fold upon itself during placement.

With the nasal cavity bandage dressing and use thereof, an inner application surface of the bandage is coated with the adhesive, including an acrylate that is safe for direct application to a nasal mucous membrane tissue, allowing for high bond strength in a wet environment with immediate functional stress.

With the nasal cavity bandage dressing and use thereof, the multi-layered absorbent sterile dressing is non-adherent to a nasal mucous membrane.

With the nasal cavity bandage dressing and use thereof, the multi-layered absorbent sterile dressing is formed with a porous polymer, including aloe vera.

With the nasal cavity bandage dressing and use thereof, the bandage is made from a clear or translucent material.

Still other aspects, features, and advantages of the present invention are readily apparent from the following detailed description, by illustrating a number of illustrative embodiments and implementations, including the best mode contemplated for carrying out the present invention. The present invention is also capable of other and different embodiments, and its several details can be modified in various respects, all without departing from the spirit and scope of the present invention. Accordingly, the drawings and descriptions are to be regarded as illustrative in nature, and not as restrictive.

### BRIEF DESCRIPTION OF THE DRAWINGS

The embodiments of the present invention are illustrated by way of example, and not by way of limitation, in the figures of the accompanying drawings and in which like reference numerals refer to similar elements and in which:

FIGS. 1A-1B are illustrative diagrams depicting an exploded perspective view of an adhesive application surface of a nasal cavity bandage dressing and placement thereof within a nasal cavity, according to an embodiment of the present invention;

FIG. 2 is diagram that depicts a side profile view of the nasal cavity bandage dressing of FIGS. 1A-1B, according to an embodiment of the present invention;

FIG. 3 is a diagram that depicts a top planar view of the nasal cavity bandage dressing of FIGS. 1A-1B, with a partially peeled back view of the adhesive surface, according to an embodiment of the present invention;

FIG. 4 is a diagram that depicts a top planar view of the nasal cavity bandage 102 of FIGS. 1-3, with a small outer non-adhesive ring along the edge of the applied surface of the bandage, according to an embodiment of the present invention;

FIGS. 5A-5B are illustrative diagrams depicting a top planar view of the nasal cavity bandage dressing of FIGS. 1-4, with a non-adhesive tab extending from the bandage and placement thereof within the right nasal cavity, according to an embodiment of the present invention; and

FIG. 6 is a diagram that depicts a top planar view of the nasal cavity bandage 102 of FIGS. 1-5, with raised bumps 112 covering the outside of the non-applied surface of the bandage 102, according to an embodiment of the present invention.

### DETAILED DESCRIPTION OF THE PREFERRED EMBODIMENTS

An illustrative bandage dressing system and method are described. In the following description, for purposes of explanation, numerous specific details are set forth in order to provide a thorough understanding of the present invention. It is apparent to one skilled in the science, however, that the present invention may be practiced without these specific details or with an equivalent arrangement or with one module deployed in the absence of the other. In some instances, well-known devices and structures are shown in block diagram in order to avoid unnecessarily obscuring the present invention. The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. Unless otherwise defined, all terms (including technical and scientific terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

The present invention includes recognition of the problem of nasal discharge, or more commonly known as a runny nose. Regular breathing of air inside and outside of the lungs is a daily activity that most people rarely take time to think about. The ease or discomfort of breathing has a major impact on the quality of our lives. The nose functions primarily as the organ of smell and as a passage through which air travels on its way in and out of the lungs. The external openings to the nose are the nostrils, which lead into two nasal cavities that are separated by the nasal wall. Mucus-lined, curved, bony projections, call turbinates, form the lateral walls of the nasal cavities and provide a large surface area for warming, humidifying, and filtering inhaled air. Each turbinate has an opening for draining the nasolacrimal ducts and four paranasal sinuses. The sinuses are air-filled spaces lined with mucous membrane that is continuous with that of the nose. Their primary purpose is to produce mucus for the nasal cavity. Nasal discharge results when there is excess mucus collecting in the nose.

Nasal discharge can disrupt clear airflow and make breathing very difficult for many persons suffering from various conditions. These conditions include, but are not limited to the following: allergies, a cold or flu virus, pregnancy (e.g., higher levels of estrogen during pregnancy causes swelling in the mucous membrane), fumes from chemicals or other irritants, dry climates, spicy foods, and sinus infections such as rhinosinusitis, also known as coryza, which is irritation and swelling of the mucous membrane inside the nose and the paranasal sinuses that lead and drain into the nose. The skin tissue surrounding the outer nose is easily irritated during acute attacks from these conditions. A runny nose can painfully irritate these delicate tissues inside and around the nose, leading to severely dry and chapped skin from the constant trauma of blowing and wiping. In addition, persons can experience increased sneezing, nasal soreness, headache, and signs of respiratory distress such as increased restlessness, abnormal breathing pattern, increased respiratory rate, complaints of dyspnea (e.g., difficulty breathing), and/or myalgia (e.g., muscular pain caused by excessive use of the accessory muscles used for breathing). Furthermore, this problem can easily and effectively spread massive amounts of germs, placing oneself and others' health that are in close contact at high risk for infection.

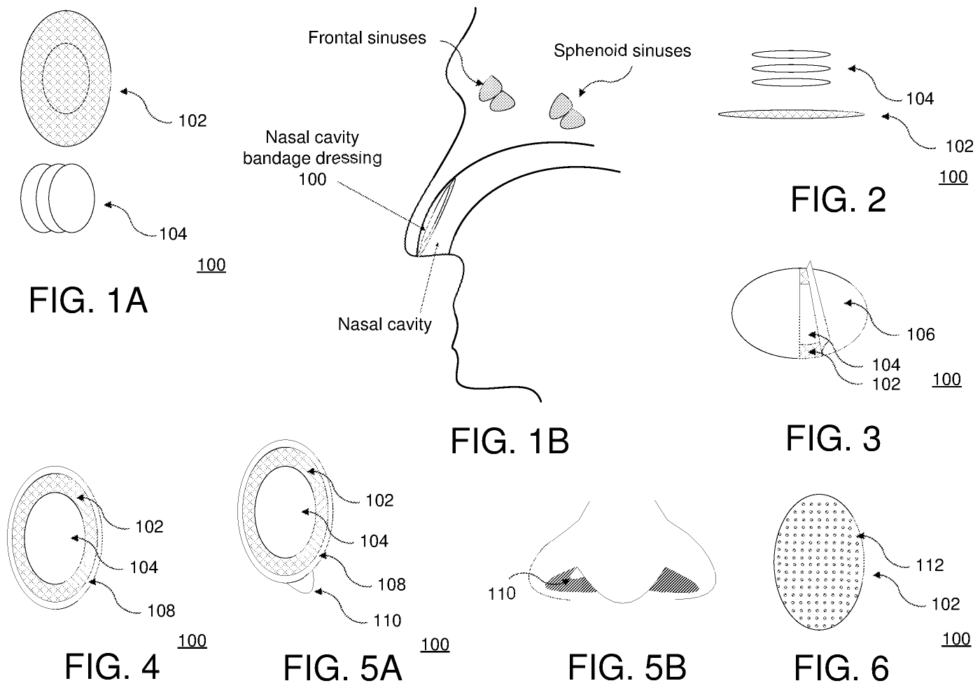

Referring now to the figures, wherein like reference numerals designate identical or corresponding parts throughout the several views, and more particularly in FIGS. 1A-1B thereof there is illustrated an exploded perspective view of an adhesive application surface of a nasal cavity bandage dressing and placement thereof within a nasal cavity, according to an embodiment of the present invention. In FIGS. 1A-1B, the nasal cavity bandage dressing 100 including a bandage 102 that can be made from a clear or translucent, waterproof antimicrobial adhesive material, and the like, that is safe for direct application to a mucous membrane, and the like.

The nasal cavity bandage can be configured using plastics, latex, moleskin, foam, silicon or other sufficiently durable and malleable material that allow for easy and sustained placement within a small space of a nasal cavity. The adhesive can be made of acrylate, and the like, which is safe for direct application to nasal mucous membrane tissue, allowing for high bond strength in a wet environment with immediate functional stress.

A crosshatch pattern is used in FIGS. 1A-1B to display the inner adhesive and application surface of the bandage. A dashed oval line is used to illustrate the placement of the sterile dressing 104 to the application side of the bandage 102. The sterile dressing 104 can also be oval in shape, but can be smaller in length and width as compared to the bandage covering. The sterile dressing 104 can be configured with thin multi-layered cotton-like absorbent sterile pads, and the like (e.g., three layers are depicted in FIG. 1). The dressing 104 can be coated with a porous polymer, such as aloe vera, and the like, to allow for soothing comfort and gentle removal.

Together, the clear or translucent, waterproof, antimicrobial bandage and sterile dressing coated with a porous polymer compose the embodiment of the nasal cavity bandage dressing. Advantageously, this bandage dressing is positioned and adhered along the immediate and upper inside of the nasal cavity to stop nasal discharge flow from the paranasal sinuses and nasolacrimal ducts.

The nasal cavity space is a highly vulnerable environment, wherein the entry and placement of any object is likely to introduce foreign pathogens (e.g., bacteria and viruses) capable of causing disease and illness. Advantageously, the nasal cavity bandage 102 is coated with an antibacterial agent, such as Benzalkonium Chloride 0.1%, and the like, to protect against the risk of infection, and the like, associated with placement of the nasal cavity bandage.

The nasal cavity is a naturally moist environment. Accordingly, the nasal cavity bandage 102 is configured using waterproof material, and the like. Advantageously, this allows for sustained placement of the bandage within a moist environment, without becoming overly saturated, and thus, useless for preventing nasal discharge.

Many persons continue to go about their normal daily activities while experiencing the onset of a runny nose. For example, many persons will continue to live active lifestyles, including going to work, dining in public restaurants, and attending events in public spaces all while interacting with others. Advantageously, the discreet and unnoticeable wear of a product placed in the nose to stop nasal discharge allow a user to continue with normal daily activities without the threat of embarrassment while engaging in public spaces.

FIG. 2 is diagram that depicts a side profile view of the nasal cavity bandage dressing of FIGS. 1A-1B, according to an embodiment of the present invention. In FIG. 2, the bandage 102 and dressing 104 materials can employ a thin configuration so that placement of the bandage does not occlude nasal passages from a clear flow of air in and outside of the nose.

FIG. 3 is a diagram that depicts a top planar view of the nasal cavity bandage dressing of FIGS. 1A-1B, with a partially peeled back view of the adhesive surface, according to an embodiment of the present invention. In FIG. 3, the nasal cavity bandage dressing 104 of FIGS. 1A-1B, includes a protective cover 106, and depicts a partially peeled back view of the adhesive surface of the bandage 102. As the preferred nasal cavity bandage 102 described herein includes an adhesive layer, it is appropriate to include the protective cover 106 to ensure proper protection of the application surface of the bandage 102, while it is packaged for consumer use. The nasal cavity bandage 102 is described in FIG. 3 in terms of the applied surface of the bandage 102 and the dressing 104 being covered by the cover 106, for example, a waxy material to serve as a protective covering. However, the protective cover 106 can be made with any suitable material that allows for proper protection of the application surface of the bandage 102. Thus, the cover 106 serving as a protective layer can be formed using appropriately sufficient materials, for example, including paraffin wax, vegetable wax, blended wax, and/or from water-soluble and water-insoluble polymers, and the like. Advantageously, the protective cover 106, serving as a protective layer can safeguard additional materials affixed to the dressing 104, such as various cosmetic fragrances, transdermal medications, aromatic medications, and the like.

FIG. 4 is a diagram that depicts a top planar view of the nasal cavity bandage 102 of FIGS. 1-3, with a small outer non-adhesive ring along the edge of the applied surface of the bandage, according to an embodiment of the present invention. In FIG. 4, the nasal cavity bandage 102 of FIGS. 1-3, includes a small outer non-adhesive ring 108 along the edge of the applied surface of the bandage 102. Although the nasal cavity bandage 102 is described in FIGS. 1-2 in terms of being coated with an adhesive material which covers a majority of the application surface, the adhesive can be stopped short on the application surface, allowing for a small outer non-adhesive ring 108 along the edge of the applied surface of the bandage 102. In doing so, the user can place the bandage dressing in the nasal cavity, and when desired, can easily remove the bandage 102 by peeling back from any edge of the outer portion of the bandage 102.

Small child users can be less concerned with discreetness of bandage dressing placement in the nostrils. In addition, it can be more desirable for parents or caregivers of users to have a visible material included as part of the bandage dressing to remind the parent or caregiver of the presence of the bandage. Accordingly, FIGS. 5A-5B are illustrative diagrams depicting a top planar view of the nasal cavity bandage dressing of FIGS. 1-4, with a non-adhesive tab extending from the bandage and placement thereof within the right nasal cavity, according to an embodiment of the present invention. In FIGS. 5A-5B, the nasal cavity bandage 102 of FIGS. 1-4, includes a non-adhesive tab 110 extending from the bandage 102. The nasal cavity bandage 102 can be designed to include the tab 110, for example, formed as a half-circle, colored-tab, and the like, advantageously allowing parents or caregivers of small child users to visibly see the presence of the bandage 102 in the nasal cavity the child, thus, reminding them of the placement of the bandage 102, and a need to remove the bandage 102 when desired.

Although the nasal cavity bandage dressing is described in FIGS. 5A-5B in terms of including a half-circle, colored visible tab 110 as a visual signal for others to be reminded of placement within the user's nasal cavity, a visual signal component can be designed with separate materials from the bandage 102, and with other tab shapes of various sizes, colors, and the like, based on the teachings of the present disclosure, as will be appreciated by those of ordinary skill in the relevant art(s).

FIG. 6 depicts the nasal cavity bandage 102 of FIGS. 1-5, with raised bumps 112 as illustrated by numerous small gray dots covering the non-applied outside surface of the bandage 102. Although the nasal cavity bandage 102 is described in FIGS. 1-5 in terms of being made from a tough, but malleable material that allows for elasticity without being flimsy so as not to fold upon itself during placement, the bandage 102 may also include raised bumps 112, and the like on the outside of the non-application surface, allowing for a more secured finger-hold of the bandage 102 during position and placement of the bandage dressing 100. In doing so, the user may position and place the bandage dressing 100 in the nasal cavity with greater accuracy and stability.

Thus, the present invention includes a nasal cavity bandage made of clear or translucent, waterproof, antimicrobial adhesive material that is safe for direct application to a mucous membrane. Attached to the bandage is a non-adherent multi-layered absorbent sterile dressing. The dressing is non-adherent to the mucous membrane and coated with a porous polymer, such as aloe vera, to allow for soothing comfort and gentle removal. The nasal cavity bandage dressing is tough, but malleable, and thus, capable of lining the inner contours of the nose with ease of placement where it can block excessive and continuous nasal discharge caused by various conditions. Advantageously, the nasal cavity bandage dressing is of a compact and clear or translucent design that is: not easily visible, does not obstruct breathing through the nose, can easily be applied with skin safe adhesive material to a nasal mucous membrane, and protects against infection while reducing the further spread of germs.

While preferred materials for elements have been described, the product is not limited by these materials. Accordingly, any suitable plastics, latex, moleskin, foam, silicon, cotton, acrylates, hypo-allergenic cosmetics, aloe vera, Benzalkonium Chloride 0.1%, and the like, can be employed in the nasal cavity bandage dressing according to further embodiments and the teachings of the present invention, as will be appreciated by those of ordinary skill in the relevant art(s).

Although the nasal cavity bandage dressing is described in FIGS. 1-2 in terms of being oval-shaped, the nasal cavity bandage dressing can be designed to take on any suitable shapes, such as diamond-shaped, square-shaped, circular-shaped, and the like, based on the teachings of the present disclosure, as will be appreciated by those of ordinary skill in the relevant art(s).

Although the nasal cavity bandage dressing is described in FIGS. 1-2 in terms of the bandage being made from a clear or translucent material, the bandage can be made of any suitable material, such as non-clear or non-translucent materials, and the like, based on the teachings of the present disclosure, as will be appreciated by those of ordinary skill in the relevant art(s).

Although the nasal cavity bandage dressing is described in FIGS. 1-2 in terms of the bandage and dressing being coated with antimicrobial or porous polymer, the bandage dressing can be coated with any suitable material, such as fragrances, medications, vitamins, and the like, for example, including fragrances, such as citrus fruits, eucalyptus, peppermint, and the like, as well as medications with pharmaceutical benefits, including Menthol, and the like, which can be used as a cooling agent, and the like, or any suitable mixture thereof, based on the teachings of the present disclosure, as will be appreciated by those of ordinary skill in the relevant art(s).

While the present invention has been described in connection with a number of embodiments and implementations, the present invention is not so limited but rather covers various modifications and equivalent arrangements, which will fall within the purview of the appended claims.

What is claimed is:

1. A nasal cavity bandage dressing, comprising:
a bandage made from a waterproof, antimicrobial, and adhesive material; and
a multi-layered, absorbent sterile dressing affixed to the bandage;
wherein the bandage affixed with the non-adherent multi-layered absorbent sterile dressing is configured to fit within a nasal cavity.

2. The nasal cavity bandage dressing of claim 1, wherein an outer surface of the bandage is coated with an antibacterial agent, including Benzalkonium Chloride 0.1% to protect against risk of infection associated with placement within the nasal cavity.

3. The nasal cavity bandage dressing of claim 1, wherein the bandage is waterproof, allowing for sustained placement within a moist environment of the nasal cavity.

4. The nasal cavity bandage dressing of claim 1, wherein the bandage is made from a tough, but malleable material that allows for elasticity without being flimsy so as not to fold upon itself during placement.

5. The nasal cavity bandage dressing of claim 1, wherein an inner application surface of the bandage is coated with the adhesive, including an acrylate that is safe for direct application to a nasal mucous membrane tissue, allowing for high bond strength in a wet environment with immediate functional stress.

6. The nasal cavity bandage dressing of claim 1, wherein the multi-layered absorbent sterile dressing is non-adherent to a nasal mucous membrane.

7. The nasal cavity bandage dressing of claim 1, wherein the multi-layered absorbent sterile dressing is formed with a porous polymer, including aloe vera.

8. The nasal cavity bandage dressing of claim 1, wherein the bandage is made from a clear or translucent material.

9. A method for using a nasal cavity bandage dressing, the method comprising
making a bandage from a waterproof, antimicrobial, and adhesive material;
affixing a multi-layered, absorbent sterile dressing to the bandage; and
configuring the bandage affixed with the non-adherent multi-layered absorbent sterile dressing to fit within a nasal cavity.

10. The method of claim 9, further comprising:
coating an outer surface of the bandage with an antibacterial agent, including Benzalkonium Chloride 0.1% to protect against risk of infection associated with placement within the nasal cavity.

11. The method of claim 9, wherein the bandage is waterproof, allowing for sustained placement within a moist environment of the nasal cavity.

12. The method of claim 9, further comprising:
making the bandage from a tough, but malleable material that allows for elasticity without being flimsy so as not to fold upon itself during placement.

13. The method of claim 9, further comprising:
coating an inner application surface of the bandage with the adhesive, including an acrylate that is safe for direct application to a nasal mucous membrane tissue, allowing for high bond strength in a wet environment with immediate functional stress.

14. The method of claim 9, wherein the multi-layered absorbent sterile dressing is non-adherent to a nasal mucous membrane.

15. The method of claim 9, further comprising:
forming the multi-layered absorbent sterile dressing with a porous polymer, including aloe vera.

16. The method of claim 9, wherein the bandage is made from a clear or translucent material.
